(19) United States
(12) Patent Application Publication
(10) Pub. No.: US 2025/0101351 A1
(43) Pub. Date: Mar. 27, 2025

(54) IN VITRO METHODS OF PRODUCING AND ASSAYING 13CO2 GAS FROM 13C-LABELED BIOMASS

(71) Applicant: Advanced Breath Diagnostics, LLC, Brentwood, TN (US)

(72) Inventors: Ethan S. McClain, Franklin, TN (US); Nicole L. Giffin, Gallatin, TN (US); Catherine Williams, Fairview, TN (US)

(21) Appl. No.: 18/888,346

(22) Filed: Sep. 18, 2024

Related U.S. Application Data
(60) Provisional application No. 63/584,318, filed on Sep. 21, 2023.

(51) Int. Cl. C12F 3/02; C12N 1/18; C12N 9/26; C12N 9/36

(52) U.S. Cl. CPC C12F 3/02; C12N 1/18; C12N 9/2414; C12N 9/2462; C12Y 302/01001; C12Y 302/01017

(57) ABSTRACT

The present invention relates to an in vitro method for producing ¹³CO₂ gas from ¹³C-labeled biomass. The invention also relates to an in vitro method of quantifying ¹³CO₂ gas produced from ¹³C-labeled biomass, and an in vitro method for determining whether a source of ¹³C-labeled biomass will produce a predictable amount of ¹³CO₂ gas during in vivo digestion. The ¹³C-labeled biomass can be ¹³C-labeled Spirulina platensis in some cases.

### RELATED APPLICATION

The present application claims priority to U.S. provisional patent application No. 63/584,318, the entire contents of which are incorporated herein by reference.

### FIELD OF THE INVENTION

The present invention generally relates to an in vitro method of producing ¹³CO₂ gas from biomass that is labeled with ¹³C. The invention also relates to an in vitro method of assaying ¹³CO₂ gas produced from ¹³C-labeled biomass.

### BACKGROUND OF THE INVENTION

Biomass that is labeled with a stable isotope is often used to analyze various pathways in human subjects to assess the presence of a disorder. One useful isotope is carbon-13 (“¹³C”) because it is present in all organic material and is non-radioactive. As a result, ¹³C-labeled biomass is often used in breath testing.

Breath testing can be used to assess gastroparesis, a disorder that slows or stops movement of food from the stomach to the small intestine. Gastroparesis can be diagnosed by measuring the rate at which a meal empties from the stomach and enters the small intestine (the “gastric emptying rate”). In breath testing, a subject ingests a test meal including ¹³C-labeled biomass such as ¹³C-labeled Spirulina platensis. The ¹³C-labeled biomass passes through the stomach, is absorbed by the small intestine, and is metabolized by the liver to give rise to ¹³CO₂. The ¹³CO₂ then moves through the blood to the lungs and exits the body through the subject's breath.

A test administrator collects breath samples from the subject before and after ingestion of the test meal. Samples are collected after ingestion at a number of different time points. The breath samples are then analyzed using a mass spectrometer, infrared spectrometer, or any other known instrument to obtain a ratio of ¹³CO₂/¹²CO₂. This ratio is used to calculate the CO₂ excretion rate. By measuring the change in excretion rate over time, the subject's gastric emptying rate can be determined.

¹³C-labeled biomass can be obtained by, growing the biomass in a medium enriched in ¹³C. Often times, different lots of ¹³C-labeled biomass are grown and harvested according to a standardized protocol. For example, the standardized protocol can specify a specific day on which the ¹³C-labeled biomass should be harvested. Additionally, in many cases, validation studies have been previously performed to show that a specific dose of ¹³C-labeled biomass grown according to the standardized protocol yields a predictable amount of ¹³CO₂ in a normal subject's breath after digestion. Therefore, test administrators assume that the amount of ¹³C administered is precisely known. This is important because breath testing results are based on the amount of ¹³CO₂ produced, which is directly related to the amount of ¹³C originally ingested.

Often, ¹³C-labeled biomass is grown in bulk according to different lots. If the standardized protocol is not followed for a given lot, ¹³C-labeled biomass from that lot may not have the desired precise amount of ¹³C and will not produce the same predictable amount of ¹³CO₂. This may lead to inaccurate breath testing results. As an example, if the ¹³C-labeled biomass is harvested too soon or too late, it may produce amounts of ¹³CO₂ in a normal subject's breath after digestion that is different than the predictable, validated amount of ¹³CO₂ from ¹³C-labeled biomass grown according to the standardized protocol.

It would be desirable to provide an in vitro method of producing ¹³CO₂ gas from ¹³C-labeled biomass that is consistent with a normal subject's in vivo digestion and respiration. It would also be desirable to provide an in vitro method of assaying ¹³CO₂ gas produced from a given lot of ¹³C-labeled biomass. Such an assay could help provide a quality control method to verify that a given lot of ¹³C-labeled biomass was grown according to a standardized protocol and will produce predictable amounts of ¹³CO₂ gas in a normal subject's breath after digestion.

### BRIEF DESCRIPTION OF THE DRAWINGS

FIG. 1 is a flow chart depicting an in vitro method for producing ¹³CO₂ gas from ¹³C-labeled biomass according to certain embodiments.

FIG. 2 is a flow chart depicting a method for assessing gastric emptying in a subject according to certain embodiments.

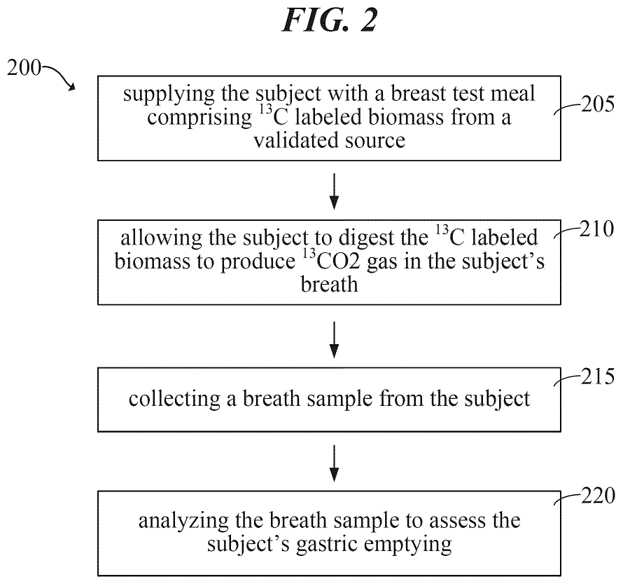

### SUMMARY OF THE INVENTION

Certain embodiments provide an in vitro method for producing ¹³CO₂ gas from ¹³C-labeled biomass. The method can comprise steps of (a) preparing a solution comprising the ¹³C-labeled biomass and one or more enzymes, (b) allowing the one or more enzymes to break down the ¹³C-labeled biomass into ¹³C-labeled simple sugars, (c) adding a yeast formulation to the solution, and (d) allowing the yeast formulation to digest the ¹³C-labeled simple sugars in the solution to produce ¹³CO₂ gas. In some cases, the step of allowing the one or more enzymes to break down the ¹³C-labeled biomass into ¹³C-labeled simple sugars includes incubating the solution for an incubating time period sufficient to enable the one or more enzymes to break down the ¹³C-labeled biomass into ¹³C-labeled simple sugars. The incubating time period can be in a range, for example a range of from 8 hours to 36 hours, such as 24 hours. In some cases, the method can include a step of heating the solution for a denaturing time period and at a denaturing temperature sufficient to denature the one or more enzymes before the step of adding the yeast formulation to the solution. The denaturing temperature can also be in a range, for example a range of from 75° C. to 150° C., such as 100° C. Also, the denaturing time period can be in a range, for example a range of from 30 minutes to 4 hours, such as 1 hour. Further, in some cases, the step of adding the yeast formulation to the solution can include adding a combination comprising both the yeast formulation and sucrose. In certain cases, the combination comprises a ratio of the yeast formulation to the sucrose in a range, for example a range of from 2:1 to 4:1, such as 3:1. The ¹³C-labeled biomass can be ¹³C-labeled Spirulina platensis in some examples. Also, in some examples, the yeast formulation can be active dry yeast obtained from Saccharomyces cerevisiae. Further, in some cases, the one or more enzymes can include α-amylase and lysozyme. In certain cases, the solution can comprise a ratio of α-amylase to lysozyme in a range, for example a range of from 0.9:1.1 to 1.1:0.9, such as 1:1. Also, in some cases, the solution can comprise a ratio of the ¹³C-labeled biomass, e.g., ¹³C-labeled Spirulina platensis, to the one or more enzymes, e.g. α-amylase and lysozyme, in a range, for example a range of from 3:1 to 6:1, such as 5:1.

Other embodiments provide an in vitro method for producing ¹³CO₂ gas from ¹³C-labeled biomass. The method can comprise the steps of (a) preparing a solution comprising the ¹³C-labeled biomass, one or more enzymes, and water, (b) incubating the solution for an incubation time period sufficient to enable the one or more enzymes to break down the ¹³C-labeled biomass into ¹³C-labeled simple sugars, (c) heating the solution for a denaturing time period and at a denaturing temperature sufficient to denature the one or more enzymes, (d) separating the solution to obtain a supernatant, (e) diluting the supernatant with water to obtain a supernatant solution, (f) warming the supernatant solution to a warming temperature and maintaining the supernatant solution at the warming temperature, (g) adding the yeast formulation and sucrose to the supernatant solution, and (h) allowing the yeast formulation to digest the ¹³C-labeled simple sugars in the supernatant solution to produce ¹³CO₂ gas. The incubating time period can be in a range, for example a range of from 8 hours to 36 hours, such as 24 hours. Also, the denaturing temperature can be in a range, for example a range of from 75° C. to 150° C., such as 100° C. Further, the denaturing time period can be in a range, for example a range of from 30 minutes to 4 hours, such as 1 hour. Even further, the warming temperature can be in a range, for example a range of from 36° C. to 42° C., such as 37° C. In some examples, the ¹³C-labeled biomass can be ¹³C-labeled Spirulina platensis. Also, the yeast formulation can be active dry yeast obtained from Saccharomyces cerevisiae in some examples. Further, in some cases, the one or more enzymes can include α-amylase and lysozyme. In certain cases, the solution can comprise a ratio of α-amylase to lysozyme in a range, for example a range of from 0.9:1.1 to 1.1:0.9, such as 1:1. Also, in some cases, the solution can comprise a ratio of the ¹³C-labeled biomass, e.g., ¹³C-labeled Spirulina platensis, to the one or more enzymes, e.g. α-amylase and lysozyme, in a range, for example a range of from 3:1 to 6:1, such as 5:1.

Other embodiments provide a method for quantifying ¹³CO₂ gas produced by ¹³C-labeled biomass during in vitro digestion. The method can comprise the steps of: (a) preparing a solution comprising the ¹³C-labeled biomass and one or more enzymes, (b) allowing the one or more enzymes to break down the ¹³C-labeled biomass into ¹³C-labeled simple sugars, (c) adding a yeast formulation to the solution, (d) allowing the yeast formulation to digest the ¹³C-labeled simple sugars in the solution to produce ¹³CO₂ gas, (e) collecting the ¹³CO₂ gas, and (f) quantifying the ¹³CO₂ gas. In some cases, the step of allowing the one or more enzymes to break down the ¹³C-labeled biomass into ¹³C-labeled simple sugars includes incubating the solution for an incubating time period sufficient to enable the one or more enzymes to break down the ¹³C-labeled biomass into ¹³C-labeled simple sugars. The incubating time period can be in a range, for example a range of from 8 hours to 36 hours, such as 24 hours. Further, in some cases, the method can include a step of heating the solution for a denaturing time period and at a denaturing temperature sufficient to denature the one or more enzymes before the step of adding the yeast formulation to the solution. The denaturing temperature can be in a range, for example a range of from 75° C. to 150° C., such as 100° C. Also, the denaturing time period can be in a range, for example a range of from 30 minutes to 4 hours, such as 1 hour. The step of adding the yeast formulation to the solution can include adding a combination comprising both the yeast formulation and sucrose. In certain cases, the combination comprises a ratio of the yeast formulation to the sucrose in a range, for example a range of from 2:1 to 4:1, such as 3:1. The ¹³C-labeled biomass can be ¹³C-labeled Spirulina platensis in some examples. Also, in some examples, the yeast formulation can be active dry yeast obtained from Saccharomyces cerevisiae. Further, the one or more enzymes can include α-amylase and lysozyme in some cases. In certain cases, the solution can comprise a ratio of α-amylase to lysozyme in a range, for example a range of from 0.9:1.1 to 1.1:0.9, such as 1:1. Also, in specific cases, the solution can comprise a ratio of the ¹³C-labeled biomass, e.g., ¹³C-labeled Spirulina platensis, to the one or more enzymes, e.g. α-amylase and lysozyme, in a range, for example a range of from 3:1 to 6:1, such as 5:1.

Other embodiments provide an in vitro method for determining whether a source of ¹³C-labeled biomass will produce a predictable amount of ¹³CO₂ gas during digestion. The method can comprise the steps of: (a) preparing a solution comprising a sample of ¹³C-labeled biomass from the source and one or more enzymes, (b) allowing the one or more enzymes to break down the ¹³C-labeled biomass sample into ¹³C-labeled simple sugars, (c) adding a yeast formulation to the solution, (d) allowing the yeast formulation to digest the ¹³C-labeled simple sugars in the solution to produce ¹³CO₂ gas, (e) collecting the ¹³CO₂ gas, (f) quantifying the ¹³CO₂ gas to obtain a value, and (g) comparing the value to a value range previously established for ¹³C-labeled biomass that produces a predictable amount of ¹³CO₂ gas during digestion. In some cases, the step of allowing the one or more enzymes to break down the ¹³C-labeled biomass into ¹³C-labeled simple sugars can include incubating the solution for an incubating time period sufficient to enable the one or more enzymes to break down the ¹³C-labeled biomass into ¹³C-labeled simple sugars. The incubating time period can be in a range, for example a range of from 8 hours to 36 hours, such as 24 hours. Further, in some cases, the method can include a step of heating the solution for a denaturing time period and at a denaturing temperature sufficient to denature the one or more enzymes before the step of adding the yeast formulation to the solution. The denaturing temperature can be in a range, for example a range of from 75° C. to 150° C., such as 100° C. Also, the denaturing time period can be in a range, for example a range of from 30 minutes to 4 hours, such as 1 hour. Also, in some cases, the step of adding the yeast formulation to the solution can comprise adding both the yeast formulation and sucrose. In certain cases, a ratio of the yeast formulation to the sucrose can be in a range, for example a range of from 2:1 to 4:1, such as 3:1. The ¹³C-labeled biomass can be ¹³C-labeled Spirulina platensis in some examples. Also, in some examples, the yeast formulation can be active dry yeast obtained from Saccharomyces cerevisiae. Further, in some cases, the one or more enzymes can include α-amylase and lysozyme. In certain cases, the solution can comprise a ratio of α-amylase to lysozyme in a range, for example a range of from 0.9:1.1 to 1.1:0.9, such as 1:1. Also, in some cases, the solution can comprise a ratio of the ¹³C-labeled biomass, e.g., ¹³C-labeled Spirulina platensis, to the one or more enzymes, e.g. α-amylase and lysozyme, in a range, for example a range of from 3:1 to 6:1, such as 5:1.

Other embodiments provide an in vivo method of assessing gastric emptying in a subject. The method can comprise the steps of: (a) supplying the subject with a breast test meal comprising ¹³C-labeled biomass from a validated source, (b) allowing the subject to digest the ¹³C-labeled biomass to produce ¹³CO₂ gas in the subject's breath, (c) collecting a breath sample from the subject, and (d) analyzing the breath sample to assess the subject's gastric emptying. The ¹³C-labeled biomass can be from a validated source that has been validated by a method comprising: (i) preparing a solution comprising the ¹³C-labeled biomass and one or more enzymes, (ii) allowing the one or more enzymes to break down the ¹³C-labeled biomass into ¹³C-labeled simple sugars, (iii) adding a yeast formulation to the solution, (iv) allowing the yeast formulation to digest the ¹³C-labeled simple sugars in the solution to produce ¹³CO₂ gas, (v) collecting the ¹³CO₂ gas, (vi) quantifying the ¹³CO₂ gas to obtain a value, and (vii) comparing the value to a value range previously established for ¹³C-labeled biomass that produces a predictable amount of ¹³CO₂ gas during digestion. In some cases, the step of allowing the one or more enzymes to break down the ¹³C-labeled biomass into ¹³C-labeled simple sugars can include incubating the solution for an incubating time period sufficient to enable the one or more enzymes to break down the ¹³C-labeled biomass into ¹³C-labeled simple sugars. The incubating time period can be in a range, for example a range of from 8 hours to 36 hours, such as 24 hours. Further, in some cases, the method includes a step of heating the solution for a denaturing time period and at a denaturing temperature sufficient to denature the one or more enzymes before the step of adding the yeast formulation to the solution. The denaturing temperature can be in a range, for example a range of from 75° C. to 150° C., such as 100° C. Also, the denaturing time period can be in a range, for example a range of from 30 minutes to 4 hours, such as 1 hour. Also, in some cases, the step of adding the yeast formulation to the solution comprises adding both the yeast formulation and sucrose to the solution. In certain cases, a ratio of the yeast formulation to the sucrose can be in a range, for example a range of from 2:1 to 4:1, such as 3:1. The ¹³C-labeled biomass can be ¹³C-labeled Spirulina platensis in some examples. Also, in some examples, the yeast formulation can be active dry yeast obtained from Saccharomyces cerevisiae. Further, in some cases, the one or more enzymes can include α-amylase and lysozyme. In certain cases, the solution can comprise a ratio of α-amylase to lysozyme in a range, for example a range of from 0.9:1.1 to 1.1:0.9, such as 1:1. Also, in some cases, the solution can comprise a ratio of the ¹³C-labeled biomass, e.g., ¹³C-labeled Spirulina platensis, to the one or more enzymes, e.g. α-amylase and lysozyme, in a range, for example a range of from 3:1 to 6:1, such as 5:1.

### DETAILED DESCRIPTION OF PREFERRED EMBODIMENTS

The following detailed description is to be read with reference to the drawings, in which like elements in different drawings have like reference numerals. The drawings, which are not necessarily to scale, depict selected embodiments and are not intended to limit the scope of the invention. Skilled artisans will recognize that the examples provided herein have many useful alternatives that fall within the scope of the invention.

The present invention provides an in vitro method of producing ¹³CO₂ gas from ¹³C-labeled biomass. The invention also provides an in vitro method of assaying ¹³CO₂ gas produced from a given lot of ¹³C-labeled biomass. Such methods could help provide a quality control method to verify that a given lot of ¹³C-labeled biomass was grown according to a standardized protocol and contains a predictable amount of ¹³C.

¹³C-labeled biomass is grown according to methods described in U.S. Pat. No. 6,872,516, the entire contents of which are incorporated herein by reference. Generally, batches of ¹³C-labeled biomass are grown on a lot-by-lot basis and in accordance with a standardized protocol. The standardized protocol specifies an assigned day on which the ¹³C-labeled biomass should be harvested. In other words, the standardized protocol specifies how many days the ¹³C-labeled biomass should grow before harvesting. The standardized protocol can specify additional standardizations and is not limited.

Additionally, ¹³C-labeled biomass grown according to the standardized protocol has been previously validated to show that it contains a predictable amount of ¹³C. The previous validation method is not limited. In some cases, the previous validation method is an in vitro assay method as described herein for samples of ¹³C-labeled biomass certified to have been grown according to the standardized protocol.

In some cases, the validation methods include performing a gastric emptying breath test on a cohort of subjects previously determined to have normal gastric emptying rates. Exemplary breath test methods and validation methods are described in U.S. Pat. Nos. 8,231,530, 8,388,53, 8,317,718, 10,772,534 and 11,134,883, the entire contents of each of which are incorporated herein by reference.

Certain embodiments provide an in vitro method for producing ¹³CO₂ gas from ¹³C-labeled biomass. The term “biomass” as used herein includes all organisms capable of photosynthetic growth such as plant cells and microorganisms (including algae) in unicellular or multicellular form that are capable of growth in a liquid phase. The term may also include organisms modified artificially or by gene manipulation. Biomass may also be used herein to refer to the amount of living matter (as in a unit area or volume of habitat). In some embodiments, the ¹³C-labeled biomass is ¹³C-labeled algae. In certain embodiments, the ¹³C-labeled biomass is ¹³C-labeled Spirulina platensis.

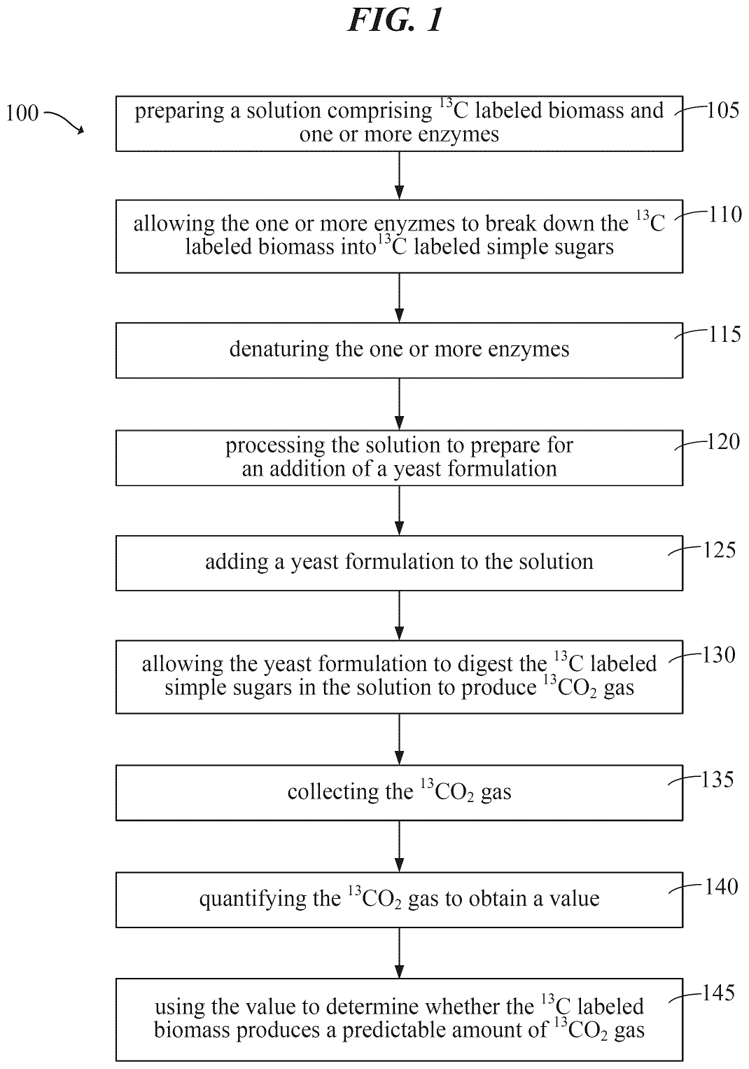

Referring to FIG. 1, an in vitro method 100 is provided. The method includes a step 105 of preparing a solution comprising ¹³C-labeled biomass and one or more enzymes. In some cases, the ¹³C-labeled biomass is ¹³C-labeled Spirulina platensis. The one or more enzymes can be selected from:
α-1,3-glucanase
α-amylase
α-glucuronidase
β-1,3-glucanase/laminarinase
β-1,3-1,4-glucanase
β-amylase
β-galactosidase
γ-amylase
Alginate lyase
Cellobiohydrolase
Cellulose 1,4-β-cellobiosidase
Chitinase 1Trans-sialidase
Chitosanase
Endo-β-2,6-fructanase
Endo-rhamnogalacturonan lyase
Exo-β-agarase
Exo-β-glucosaminidase
Keratan sulphate hydrolase/keratanase II
Laccase
Laminarinase
Lysozyme
Lytic transglycosylase
Oligoalginate lyase; and
Peptidoglycan N-acetylmuramic acid deacetylase.
   In some cases, the one or more enzymes comprises α-amylase and lysozyme.

In specific cases, the solution comprises ¹³C-labeled Spirulina platensis, a-amylase, lysozyme and water. In some cases, the solution comprises a ratio of α-amylase to lysozyme in a range, for example a range of from 0.9:1.1 to 1.1:0.9, such as 1:1. Also, in some cases, the solution comprises a ratio of Spirulina platensis to the enzymes (α-amylase and lysozyme) in a range, for example a range of from 3:1 to 6:1, such as 5:1.

Also, in some cases, the solution comprises a 10:1:1:1 ratio of Spirulina platensis to α-amylase to lysozyme to water. In particular cases, the solution comprises ˜100 mg ¹³C-labeled Spirulina platensis, 10 mg of α-amylase, 10 mg of lysozyme and ˜13 mL water.

The method also includes a step 110 of allowing the one or more enzymes to break down the cell wall of the ¹³C-labeled biomass into ¹³C-labeled simple sugars. In some cases, step 110 includes incubating the solution for an incubating time period sufficient to enable the one or more enzymes to break down the ¹³C-labeled biomass into ¹³C-labeled simple sugars. The incubating time period can be a time period in range, for example a range of 8 to 36 hours, such as 24 hours.

Next, a step 115 of denaturing the enzymes is performed. In some cases, step 115 is performed by heating the solution for a denaturing time period and at a denaturing temperature sufficient to denature the enzymes. The denaturing time period can be a time period in the range, for example a range of 30 minutes to 4 hours, such as 1 hour. Likewise, the denaturing temperature can be a temperature in a range, for example a range of from 75° C. to 150° C., such as 100° C. In specific cases, the solution is placed in a 100° C. oven for 1 hour.

The method also includes a step 120 of processing the solution to prepare for an addition of a yeast formulation. In some cases, the processing step 120 includes separating the solution to remove cellular debris and leaving behind a supernatant that contains the ¹³C-labeled simple sugars. In certain cases, the solution is provided in a centrifuge tube and provided in a centrifuge to perform the separation.

The processing step 120 can also include adding water to the supernatant. In some cases, water is added so that the total volume of supernatant and water in the solution is 100 mL. Further, the processing step can also include heating the solution to a temperature that activates the yeast formulation. In some cases, the heating is performed by positioning a container holding the solution in a water bath to bring the solution to a temperature within a range, for example a range of from 35° C. to 45° C., such as 37° C.

Further, the method includes a step 125 of adding a yeast formulation to the solution. In some cases, the yeast formulation is active dry yeast obtained from Saccharomyces cerevisiae. In certain cases, the step 125 includes adding both the yeast formulation and sucrose to the solution. In some cases, the solution comprises a 2:1 ratio of water to Saccharomyces cerevisiae in mL:g. Also, in some cases, the solution comprises a ratio of Saccharomyces cerevisiae to sucrose in a range, for example a range of from 2:1 to 4:1, such as 3:1. In specific cases, 6 g dry active Saccharomyces cerevisiae and 2.5 g of sucrose are added.

Next is a step 130 of allowing the yeast formulation to digest the ¹³C-labeled simple sugars in the solution to produce ¹³CO₂ gas. In some cases, the step 130 includes maintaining the solution at the temperature that activates the yeast formulation. In some cases, the solution is maintained in the water bath at a temperature of approximately 37° C. During this time, the yeast digests the ¹³C-labeled simple sugars to produce ¹³CO₂ gas.

Some embodiments provide a method for quantifying ¹³CO₂ gas produced by ¹³C-labeled biomass during in vitro digestion. The method includes steps 105-130 as discussed above. The method further includes a step 135 of collecting the ¹³CO₂ gas and a step 140 of quantifying the ¹³CO₂ gas. The step 135 of collecting the ¹³CO₂ gas can be performed using any gas collection device or gas collection method known in the art. In some cases, a gas collection bag is secured to the container holding the solution so that gas becomes trapped in the bag. Additionally, the gas collection bag can include a one-way value to enable one to extract gas from the bag using a syringe or like object.

The step 140 of quantifying the ¹³CO₂ gas can also be performed using a mass spectrometer, infrared spectrometer or any other known instrument to obtain a value. In certain cases, the gas sample is analyzed using a Gas Isotope Ratio Mass Spectrometer (GIRMS) to provide a sample ratio (R_{B}) of ¹³CO₂/¹²CO₂. At the same time, a reference sample having a known ¹³C abundance is analyzed with the GIRMS to provide a reference ratio (R_{S}) of ¹³CO₂/¹²CO₂. Next, a “delta per mil” difference between the ¹³CO₂/¹²CO₂ of R_{B} and R_{S} is calculated. The delta per mil value is expressed as ‰.

Finally, an additional step 145 includes using the value obtained in step 140 to determine whether the ¹³C-labeled biomass produces a predictable amount of ¹³CO₂ gas during digestion. In some cases, the value is compared to a previously established value range that indicates that the ¹³C-labeled biomass produces a predictable amount of ¹³CO₂ gas during digestion.

Other embodiments provide an in vivo method 200 of assessing gastric emptying in a subject. The method 200 includes a step 205 of supplying the subject with a breast test meal comprising ¹³C-labeled biomass from a validated source. The validated source can be a source of ¹³C-labeled biomass wherein a sample of ¹³C-labeled biomass from that source has been validated using any of the methods described herein. In some cases, the source is a lot of ¹³C-labeled biomass grown in a single bioreactor system. In specific cases, the source is a lot of ¹³C-labeled biomass grown in a single carboy of a bioreactor system.

The breath test meal includes ¹³C-labeled biomass from a validated source. In some cases, the test meal includes a ¹³C, label incorporated into Spirulina platensis. Spirulina platensis labeled with ¹¹C can be obtained by growing the algal cells in a ¹³C-enriched environment as is disclosed in commonly assigned U.S. Pat. No. 6,872,516, the disclosure of which is herein incorporated by reference in its entirety.

The validated ¹³C-labeled biomass includes the ¹³C label at a predictable dose. In some cases, the dose is between about 20 mg and about 80 mg of ¹³C label or perhaps between about 40 mg and about 50 mg, such as 43 mg of ¹³C. In certain cases, the subject ingests ¹³C-labeled Spirulina platensis at a dose of approximately 100 mg, which contains approximately 43 mg of ¹³C.

The ¹³C-labeled biomass is also incorporated into an edible food component that forms part of the test meal. The test meal includes any number of edible food components that can be ingested at a single setting. A single setting can be a designated time period, perhaps a period of less than 30 minutes, 20 minutes, or even 10 minutes. In many cases, the test meal can include a main food component as well as any side components and/or liquid components. In certain cases, the test meal includes food components derived from standardized, freeze-dried or lyophilized food components, such as those described in U.S. Pat. No. 8,178,315 or 8,753,609 the entire contents of each of which are incorporated herein by reference.

The method 200 also includes steps 205 of allowing the subject to digest the ¹³C-labeled Spirulina platensis to produce ¹³CO₂ gas in the subject's breath, a step 210 of collecting a breath sample from the subject and a step 215 of analyzing the breath sample to assess the subject's gastric emptying. In many cases, the method 200 can be performed using methods described in U.S. Pat. Nos. 8,231,530, 8,388,53, 8,317,718, 10,772,534 and 11,134,883, the entire contents of each of which are incorporated herein by reference.

The subject deposits a breath sample by blowing through a straw into the bottom of a glass tube to displace contained air and capture a clean breath sample. The test administrator caps the tube and then obtains the ¹³CO₂ content measurement of the breath for the tube using a mass spectrometer, infrared spectrometer or any other known instrument for measuring a ¹³CO₂/¹²CO₂ ratio in a gas sample.

An in vitro method of producing and quantifying ¹¹³CO₂ gas from ¹¹³C-labeled Spirulina platensis was performed as shown in steps 1 through 24 below.

### Example 1

1. Provide a 15 mL centrifuge tube.
2. Add 10 mg of α-amylase (from Aspergillus oryzae, ˜30 U/mg, MilliporeSigma) to the centrifuge tube.
3. Add 10 mg of lysozyme (from chicken egg white, >40,000 U/mg, MilliporeSigma) to the centrifuge tube.
4. Add ˜100 mg of ¹³C-labeled Spirulina platensis from an identified lot to the centrifuge tube.
5. Add ˜13 mL of USP sterile water to the centrifuge tube.
6. Incubate the centrifuge tube for 24 hours at room temperature while on a test tube rotator. This step is performed to allow the α-amylase and the lysozyme to break down the peptidoglycan cell wall of the ¹³C-labeled Spirulina platensis into ¹³C-labeled simple sugars.
7. Position the centrifuge tube in a 100° C. oven for 1 hour. This step is performed to denature the α-amylase and the lysozyme.
8. Remove the centrifuge tube from the oven and cool to room temperature.
9. Place centrifuge tube in a centrifuge. This step removes cellular debris and leave behind a supernatant.
10. Provide a 250 mL Erlenmeyer flask having a side arm and a gas collection bag secured to an opening of the side arm. The gas collection bag includes a one-way valve and is leak tight.
11. Add the supernatant to the Erlenmeyer flask.
12. Add water to the supernatant so that the total volume of supernatant and water solution in the Erlenmeyer flask is 100 mL.
13. Place the Erlenmeyer flask in a water bath and warm the solution to approximately 37° C.
14. Add 6 g dry active Saccharomyces cerevisiae (baker's yeast) to the warmed solution.
15. Add 2.5 g of sucrose to the warmed solution.
16. Seal the top of the Erlenmeyer flask with a rubber stopper.
17. Hold the Erlenmeyer flask in the water bath to keep the solution at approximately 37° C. for one hour. During this time, sample gas produced from the solution is collected in the attached gas collection bag.
18. Provide a Vacutainer tube having a 10 mL draw.
19. Use a syringe to add 9.4 mL of atmospheric air to the Vacutainer tube.
20. Insert the syringe through one-way valve on gas collection bag to draw 0.6 mL of sample gas.
21. Use the syringe to add the 0.6 mL of sample gas to the Vacutainer tube. This results in the sample gas in the Vacutainer tube having a CO₂ content of about 4%.
22. Analyze the sample gas using a Gas Isotope Ratio Mass Spectrometer (GIRMS) to provide a sample ratio (R_{B}) of ¹³CO₂/¹²CO₂.
23. Analyze a reference sample having a known isotopic abundance with GIRMS to provide a reference ratio (R_{S}) of ¹³CO₂/¹²CO₂.
24. Calculate the “delta per mil” difference between the ¹³CO₂/¹²CO₂ of R_{B} and R_{S}. The delta per mil value is expressed as ‰.

### Example 2

The in vitro method of Example 1 was performed on ¹³C-labeled Spirulina platensis obtained from 6 different lots A through F. ¹³C-labeled Spirulina platensis from lots D-F were grown according to a standard protocol that has been previously validated as growing ¹³C-labeled Spirulina platensis with a predictable amount of ¹³C and therefore produces a delta per mil value ‰ within a predictable range. ¹³C-labeled Spirulina platensis from lots A-C were grown according to a modified protocol wherein the ¹³C-labeled Spirulina platensis was harvested earlier in a final growth stage at 6 days rather than the standardized 9 days. As shown in Table 1, the results demonstrate that the day ¹³C-labeled Spirulina platensis is harvested impacts the delta per mil value ‰ as measured using the in vitro method of Example 1.

TABLE 1

| | Days in | | |
|---|---|---|---|
| Spirulina | Final Stage | Delta (‰, | |
| Lot | of Growth | n = 3) | SD |
| A | 6 | 163 | 6 |
| B | 6 | 163 | 14 |
| C | 6 | 159 | 4 |
| D | 9 | 59 | 22 |
| E | 9 | 83 | 7 |
| F | 9 | 78 | 3 |

The in vitro method of Example 1 can therefore be performed as an assay to determine whether a lot of ¹³C-labeled Spirulina platensis produces a value within a predictable range that indicates it was grown according to a desired protocol. In Example 2, a delta per mil value ‰ within a range of 150-170 indicates that a lot was harvested in a final growth stage after 6 days of growth. Similarly, a delta per mil value ‰ within a range of 50-90 indicates that a lot was harvested in a final growth stage after 9 days of growth. If the delta per mil value ‰ is outside a designated range, the lot can be disposed of for not meeting quality control standards.

### Example 3

Next, an in vivo gastric emptying breath test method was performed using ¹³C-labeled Spirulina platensis from lots A-D. This method illustrated that in vivo results from lots A-C were consistent, ¹³C-labeled Spirulina platensis from each lot was lyophilized and provided as part of a test meal. The test meal included reconstituted lyophilized whole eggs and ¹³C-labeled Spirulina platensis in an amount of 100 ng (which contained approximately 43 mg of ¹³C). The test meal also included 6 Nabisco PREMIUM saltine crackers. The test meal was used to perform an in vivo gastric emptying breath test on a cohort of normal subjects as follows:

TABLE 2

| Spiru- | | | Average kPCD (min⁻¹) (s.d.) at each time point | | | | | |
|---|---|---|---|---|---|---|---|---|
| lina | Days in | | 45 | 90 | 120 | 150 | 180 | 240 |
| Lot | Stage 4 | N | min | min | min | min | min | min |
| A | 6 | 15 | 20.3 | 54.4 | 69.4 | 79.1 | 79.6 | 67.4 |
| | | | (6.9) | (11.5) | (11.0) | (7.3) | (5.8) | (12.2) |
| B | 6 | 15 | 23.0 | 52.3 | 68.4 | 73.6 | 73.0 | 66.9 |
| | | | (6.1) | (8.6) | (9.0) | (8.7) | (9.2) | (9.5) |
| C | 6 | 15 | 22.2 | 57.6 | 74.3 | 79.3 | 78.6 | 65.6 |
| | | | (6.9) | (11.5) | (10.3) | (6.0) | (7.4) | (11.3) |
| D | 9 | 21 | 15.1 | 39.5 | 49.8 | 54.8 | 53.3 | 46.4 |
| | | | (5.4) | (9.7) | (9.4) | (8.2) | (7.3) | (7.5) |

While some preferred embodiments of the invention have been described, it should be understood that various changes, adaptations and modifications may be made therein without departing from the spirit of the invention and the scope of the appended embodiments.

1. An in vitro method for producing ¹³CO₂ gas from ¹³C-labeled biomass, comprising:
preparing a solution comprising the ¹³C-labeled biomass and one or more enzymes;
allowing the one or more enzymes to break down the ¹³C-labeled biomass into ¹³C-labeled simple sugars;
adding a yeast formulation to the solution; and
allowing the yeast formulation to digest the ¹³C-labeled simple sugars in the solution to produce ¹³CO₂ gas.

2. The method of claim 1 wherein the ¹³C-labeled biomass is ¹³C-labeled Spirulina platensis.

3. The method of claim 2 wherein the solution comprises a ratio of ¹³C-labeled Spirulina platensis to the one or more enzymes in a range of from 3:1 to 6:1.

4. The method of claim 1 wherein the yeast formulation is active dry yeast obtained from Saccharomyces cerevisiae.

5. The method of claim 1 wherein the one or more enzymes comprises α-amylase and lysozyme.

6. The method of claim 5 wherein the solution comprises a ratio of α-amylase to lysozyme in a range of from 0.9:1.1 to 1.1:0.9.

7. The method of claim 1 wherein the step of allowing the one or more enzymes to break down the ¹³C-labeled biomass into ¹³C-labeled simple sugars comprises incubating the solution for an incubating time period sufficient to enable the one or more enzymes to break down the ¹³C-labeled biomass into ¹³C-labeled simple sugars.

8. The method of claim 7 wherein the incubating time period is in a range of from 8 hours to 36 hours.

9. The method of claim 1 further comprising a step of heating the solution for a denaturing time period and at a denaturing temperature sufficient to denature the one or more enzymes before the step of adding the yeast formulation to the solution.

10. The method of claim 9 wherein the denaturing time period is in a range of from 30 minutes to 4 hours, such as 1 hour, and the denaturing temperature is in a range of from 75° C. to 150° C.

11. The method of claim 1 wherein the step of adding the yeast formulation to the solution comprises adding a combination comprising both the yeast formulation and sucrose.

12. The method of claim 11 wherein the combination comprises a ratio of the yeast formulation to the sucrose in a range of from 2:1 to 4:1.

13. An in vitro method for producing ¹³CO₂ gas from ¹³C-labeled biomass, comprising:
preparing a solution comprising the ¹³C-labeled biomass, one or more enzymes, and water;
incubating the solution for an incubation time period sufficient to enable the one or more enzymes to break down the ¹³C-labeled biomass into ¹³C-labeled simple sugars;
heating the solution for a denaturing time period and at a denaturing temperature sufficient to denature the one or more enzymes;
separating the solution to obtain a supernatant;
diluting the supernatant with water to obtain a supernatant solution;
warming the supernatant solution to a warming temperature and maintaining the supernatant solution at the warming temperature;
adding the yeast formulation and sucrose to the supernatant solution; and
allowing the yeast formulation to digest the ¹³C-labeled simple sugars in the supernatant solution to produce ¹³CO₂ gas.

14. The method of claim 13 wherein the ¹³C-labeled biomass is ¹³C-labeled Spirulina platensis.

15. The method of claim 13 wherein the yeast formulation is active dry yeast obtained from Saccharomyces cerevisiae.

16. The method of claim 13 wherein the solution comprises a ratio of the ¹³C-labeled biomass to the one or more enzymes in a range of from 3:1 to 6:1.

17. The method of claim 13 wherein the one or more enzymes comprises α-amylase and lysozyme.

18. The method of claim 17 wherein the solution comprises a ratio of α-amylase to lysozyme in a range of from 0.9:1.1 to 1.1:0.9.

19. The method of claim 13 wherein the incubating time period is in a range of from 8 hours to 36 hours.

20. The method of claim 13 wherein the denaturing time period is in a range of from 30 minutes to 4 hours, such as 1 hour, and the denaturing temperature is in a range of from 75° C. to 150° C.

21. The method of claim 13 wherein the warming temperature is in a range of from 36° C. to 42° C.

22-53. (canceled)
